(19) United States
(12) Patent Application Publication
(10) Pub. No.: US 2008/0004548 A1
(43) Pub. Date: Jan. 3, 2008

(54) Stockings for prevention and treatment of deep venous thrombosis incorporating ultrasound

(75) Inventor: Alexander Roman Oshmyansky, Littleton, CO (US)

Correspondence Address: Alexander Oshmyansky, 8712 W. Fair Dr., Littleton, CO, 80123, US

(73) Assignee: Alexander Roman Oshmyansky, Littleton, CO (US)

(21) Appl. No.: 11/821,942

(22) Filed: Jun. 26, 2007

Related U.S. Application Data
(60) Provisional application No. 60/817,582, filed on Jun. 30, 2006.

(51) Int. Cl. A61H 23/00; A61F 13/08; A61H 23/04

(52) U.S. Cl. 601/2; 601/152; 602/75

(57) ABSTRACT

A device which combines pneumatic compression therapy and ultrasound based therapy for the prevention and treatment of thrombosis. Ultrasound transducers (11) are placed in specially designed pockets (12) in stockings (23) or leggings (25). This allows for convenient use of ultrasound therapy. Pneumatic compression is also carried out by air bladders (14) which are placed in leggings. The overall result is an easy to use device which should produce significantly better results in the prevention and treatment of thrombosis, ultimately reducing patient morbidity and mortality.

### CROSS-REFERENCES TO RELATED APPLICATIONS

This application claims the benefit of provisional patent application Ser. No. 60/817,582, filed Jun. 30, 2006 by the present inventor.

### FEDERALLY SPONSORED RESEARCH

None

### SEQUENCE LISTING

None

### BACKGROUND OF THE INVENTION

1. Field of the Invention

This invention relates to the prevention and treatment of thrombosis, with a particular focus on the treatment of deep venous thrombosis (DVT) in human legs.

2. Prior Art

Deep venous thrombosis (DVT) ultimately kills hundreds of thousands of people annually in the United States alone. Blood clots in the in the deep leg veins often embolize to the pulmonary arteries coming off of the heart or to other important vessels throughout the body. Embolization can cause a variety of life threatening complications. One such complication is prevention of blood oxygenation in the lungs due to pulmonary embolism (PE). PE can be fatal if not treated promptly. However, treatment through thrombolytics or anticoagulants can be very risky as well. These medications increase the risk for stroke and other major complications. It is thus of critical need to find a mechanism that can effectively prevent or safely treat DVT.

The best way to prevent blood clots in humans and in other mammals is mobilization, i.e. walking. However, in the post-operative population, early mobilization is sometimes not possible. This induces venous stasis, one of the three components of Virchow's triad of risk factors for DVT. (The other two components of the triad are: 1) if the patient is in a state in which they are particularly prone to thrombus formation, such as if they have a cancer or if they are a smoker for example, and if 2) there is some venous trauma due to surgery, the risk for thrombus formation can increase even further.) Thus the population of patients who have recently had a surgical procedure, or those who are incapable of walking in general, is particularly vulnerable to the formation of DVT and other thrombi.

One method of preventing DVT is demonstrated in U.S. Pat. No. 5,022,387. The pneumatic compression stockings work presumably by preventing venous stasis. They are currently the only method of for the prevention of DVT which have been shown to be clinically efficacious in reducing the risk of DVT. However, the risk reduction which studies show is relatively small.

Another method of preventing and lysing thrombi is shown in U.S. Pat. No. 5,399,158. In this device, an ultrasound device is directed toward a thrombus in conjunction with a thrombolytic agent with the intention of lysing any thrombi that have formed or are beginning to form. The mechanism of action for this device is unknown. It has been hypothesized though that it may involve improved delivery of the drugs through turbulent microcurrents. However, there has yet to be any device created that would easily and effectively place an ultrasound device against the body of a mammal for any extended period of time that would allow ultrasound to be effectively administered. Ultrasonic prevention of DVT is thus not commonly used in clinical practice.

There is evidence that low-molecular-weight heparin and some other anticoagulants when administered in small doses can help prevent DVT while not significantly increasing the risk for bleeding related complications.

There is thus a great need for a device that produces a significantly reduced risk of DVT and thrombus formation in general over and above currently used devices. The drugs currently used in conjunction with ultrasound therapy (thrombolytics, etc.) are potentially dangerous. There is, insofar as I am aware, no device which effectively places an ultrasound device against a mammal for long term ultrasound based therapy and prophylaxis of thrombus formation. There is also no device which effectively combines the benefits of pneumatic compression stockings and ultrasound based therapy.

3. Objects and Advantages

Accordingly, beside the objects and advantages of the invention described in my above patent, the objects and advantages of the present invention include:
(a) Significantly improved results in the prevention of thrombosis and specifically DVT.
   (i) Secondary to the above, prevention of PE and thus significantly improved survival outcomes in hospitalized patients.
   (ii) Results improved over either compression therapy or ultrasonic therapy independently.
(b) Allowing ultrasound therapy of thrombosis to be conveniently used by health care practitioners.
(c) Miniaturizing the ultrasound providing mechanism in ultrasound therapy by eliminating the need for direct visualization of the treatment site.
(d) Allowing use of safer drugs such as low-molecular weight heparin rather than potentially more dangerous anti-embolic and thrombolytic drugs.

Further objects and advantages of my invention will become apparent form a consideration of the drawings and ensuing descriptions.

### SUMMARY

The invention is a device that combines both ultrasonic and pneumatic compression therapy for prevention and treatment of thrombosis in mammals, specifically DVT in human legs. The synergism of the two mechanisms provides improved results over either treatment modality individually. The device contains leggings/stockings which press ultrasound transducers against the legs. This allows for easy, passive application of ultrasound therapy of thrombosis which is currently preventing ultrasound therapy from being commonplace in clinical practice. Within leggings, air bladders are included to allow for pneumatic compression therapy.

### DRAWINGS—FIGURES

FIG. 1 shows a perspective view from above and the bottom-right of a legging containing both ultrasound and pneumatic components on a human leg with a standard surgical stocking underneath.

FIG. 2 shows a perspective view from above and the bottom-right of a surgical stocking containing ultrasound transducers in pockets throughout.

FIG. 3 shows a perspective view from above and the bottom-right of a pneumatic legging wrapped around a surgical stocking containing ultrasound components.

FIG. 4 shows a cross section through a pocket containing an ultrasound transducer from the side on a human leg.

FIG. 5 shows a magnified perspective view from above and the bottom-right of a pocket containing an ultrasound transducer.

### DRAWINGS—REFERENCE NUMERALS

10 Human leg
11 Ultrasound transducer
12 Pocket for transducer
13 Insulated wiring
14 Air bladder
15 Pneumatic tubing
16 Insulated wiring bundle
17 Hook-and-loop fastener
18 Surgical Stocking
19 Control Box
20 Air pump
21 Power source
22 Pocket for transducer
23 Surgical Stocking
24 Control box
25 Cloth legging
26 Air bladder
27 Pneumatic tubing
28 Ultrasound transducer-containing stocking
29 Ultrasound transducer
30 Wing attached to transducer
31 Hole in stocking
32 Patch
33 Stocking
34 Edge of hole in stocking
35 Sewing

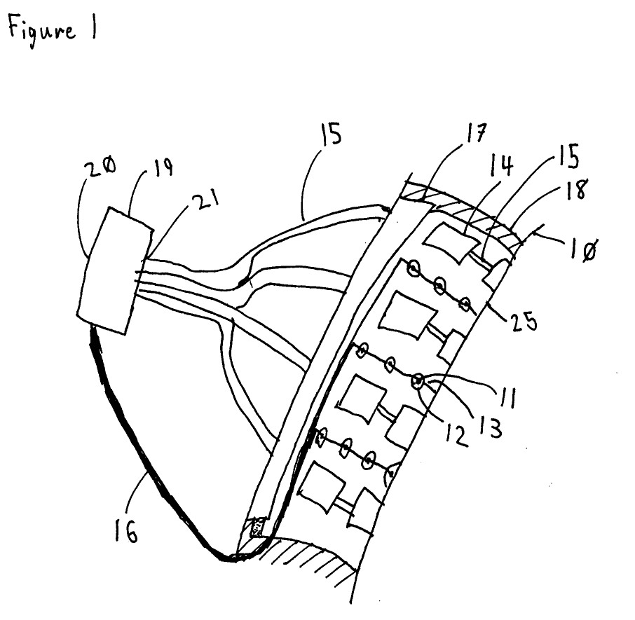

### DETAILED DESCRIPTION—FIGS. 1, 4, and 5-PREFERRED EMBODIMENT

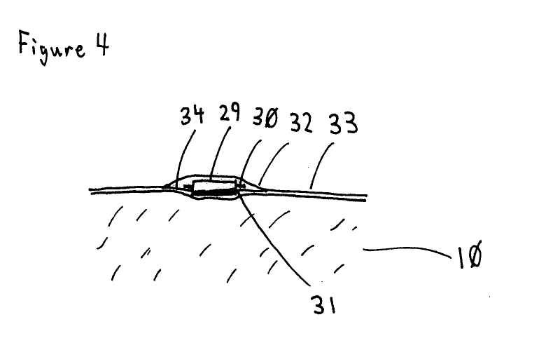

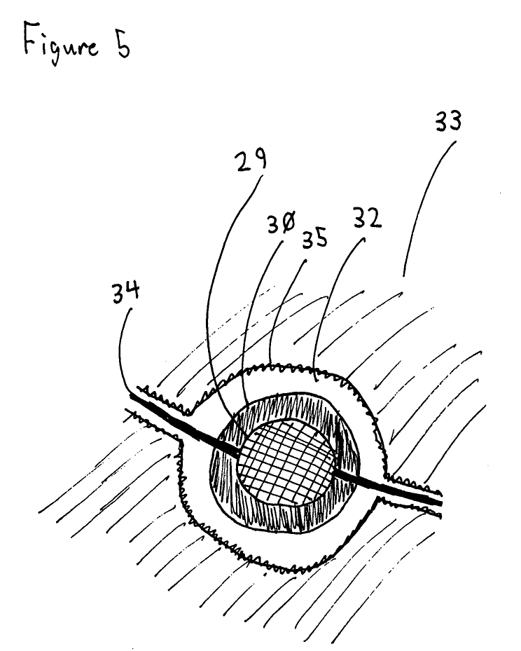

FIG. 1 is a perspective view of the invention around a single human leg, taken from a patient's bottom-right. Immediately on top of the leg, a surgical stocking 18 is placed. Wrapped on top of the leg is a cloth legging in accordance with the invention 25. It is secured with a long hook-and-loop fastener 17 which extends along the whole of the length of the legging. The hook-and-loop fastener 17 also extends several inches along the width of the legging 25 to allow for wrapping around a wide range of leg sizes. Within and underneath the legging in alternating rows are air bladders 14 and ultrasound transducers 11. I presently prefer four rows of bladders 14 and three rows of transducers illustrated though these numbers can be varied to achieve optimal results. The transducers 11 are placed within pockets 12, the potential design of which is further described in FIG. 4 and FIG. 5. I presently prefer five transducers placed around the circumference of the leg though this number can be varied to achieve optimal results. The transducers are connected by insulated wiring 13. The insulated wiring within each row 13 is then brought together in the vertical bundle of insulated wiring 16 and connected to the control box 19. The control box contains either battery power 21 or an AC power cable which is not illustrated. The air bladders 14 air connected within each row by pneumatic tubing 15. The pneumatic tubing 15 is then connected to an air pump 20 in the control box 19. I presently prefer four air bladders placed around the circumference of the leg though this number can be varied to achieve optimal results. The air bladders are sewn into pockets underneath the legging with holes in the stitching to allow for passage of pneumatic tubing to attach to each bladder.

FIG. 4 shows a cross-section through the side of an individual ultrasound transducer. The transducer is held firmly against the leg 10 which may or may not be covered by a surgical stocking. There is a circular hole 31 the diameter of a single ultrasound transducer in the legging 33. A transducer 29 is placed within this hole 31 but is prevented from falling entirely through by a wing 30 fused around the transducer. A patch 32 is attached on top of the legging to ensure that the wing is held firmly against the edges 34 of the hole 31. Thus, the transducer is placed in direct contact with the leg below. The patch also covers the insulated wiring attached to the transducer which is not shown.

FIG. 5 is a magnified view of a single pocket containing an ultrasound transducer as seen from above and to the bottom-right. As in FIG. 4, an ultrasound transducer 29 with a given diameter is placed in a hole with the same diameter. The transducer is prevented from falling through the hole by a wing 30 which is fused to the circumference of the transducer 29. A patch 32 is attached to the legging or stocking 33 to ensure that the wing is held firmly to the legging or stocking, and thus the transducer is placed in direct contact with the leg below. The patch also extends to cover the insulated wiring 34. I prefer at this time the patch be attached by sewing 35.

### OPERATION—FIGS. 1, 4, and 5—PREFERRED EMBODIMENT

FIG. 1—The air bladders 14 are periodically inflated and deflated by the air pump 20. This action, in theory, prevents venous stasis and thus helps prevent thrombosis. However, I do not wish to be bound by this theory. The rate at which this action occurs can preferably be altered and is set on the control box 19. The transducers preferably function either continuously or in pulses. The frequency, intensity, pulse rate, and mode (continuous or pulse) can preferably be varied on the control box 19. A default mode will preferably be preset for both the pneumatic and ultrasound controls however. The ultrasound waves, in theory, can allow for better circulation of pharmacological therapy thus producing significantly better results. However, I do not wish to be bound by this theory.

FIGS. 4 AND 5—The patches 32 apply pressure to a transducer 29. Each transducer 29 is placed in a hole 31 with the same diameter but is prevented from falling through by a wing 30 which is fused to the transducer. The pressure applied by the patch 32 on the transducer 29 keeps the ultrasound transducer constantly against a leg or other body part to apply the therapeutic sound waves. This allows for easy application of therapeutic ultrasound which has not yet been achieved.

The therapeutic ultrasound is preferably meant to be used in conjunction with a thrombolytic or anti-embolic agent. Preferably, a prophylactic dose of low molecular weight heparin is given prior to usage of the device. The use of a combination of ultrasound therapy, pneumatic therapy, and pharmacologic therapy should produce significantly better results then have been achieve thus far in the prevention and therapy of thrombosis and specifically DVT.

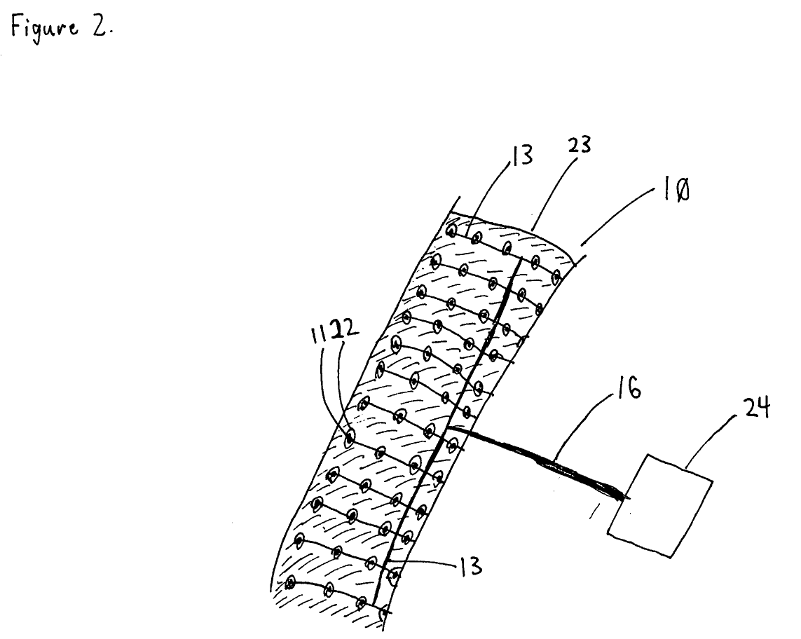

### DETAILED DESCRIPTION—FIGS. 2, 3, 4, AND 5—ALTERNATIVE EMBODIMENT

FIG. 2 is a perspective view of the bottom layer of an alternative embodiment of the invention around a single human leg 10, taken from a patient's bottom-right. In this layer of the embodiment, ultrasound transducers 11 are placed within a surgical stocking 23. The transducers are placed in pockets 22 which have the design in FIGS. 4 and 5. I presently prefer eight rows of five transducers, but this number can be altered to produce optimal results. The transducers are connected through insulated wiring 13. These wires come together into a bundle 16 which goes into a control box 24. The control box is powered either by battery or by an alternative current ground line.

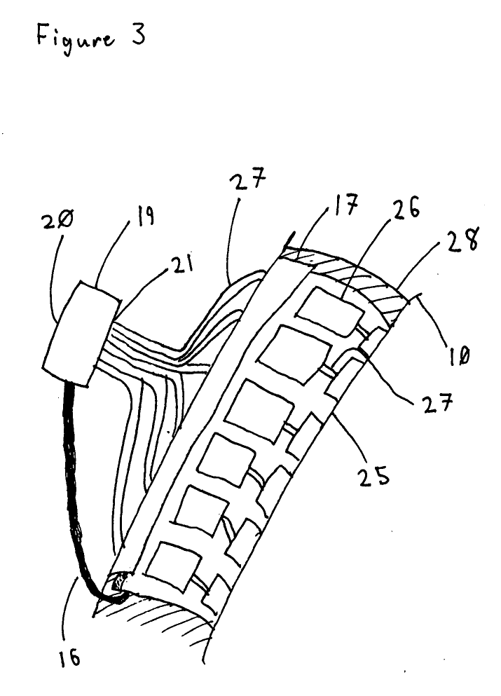

FIG. 3 is a perspective view of the top layer of an alternative embodiment of the invention around a single human leg 10, taken from a patient's bottom-right. In this layer of the alternative embodiment, a cloth legging 25 is wrapped around on top of the ultrasound-containing surgical stocking 28. The legging is secured using a hook-and-loop fastener 17 which extends along the length of legging. The hook-and-loop fastener 17 also extends several inches along the width of the legging 25 to allow for wrapping around a wide range of leg sizes. Air bladders 26 are placed within pockets attached underneath the legging. The air bladders 26 are connected by pneumatic tubing 27. Further pneumatic tubing 27 is connected to an air pump 20 in control box 19. The wiring bundle 16 from the ultrasound containing stocking 28 is also shown going into the control box 19. The power source for the invention 21, which is either a battery or an AC ground line, is also numbered in this figure.

FIG. 4 AND 5—Aside from a change in the material, the pockets in the ultrasound containing stocking 28 are designed in fundamentally the same way as is found in the preferred embodiment described above.

### OPERATION—FIGS. 2, 3, 4, AND 5—ALTERNATIVE EMBODIMENT

In contrast to the preferred embodiment, the two layer design allows for greater leg coverage by ultrasound transducers and air bladders. However, this may not be necessary. Otherwise, the operation of the alternative embodiment is fundamentally the same as in the preferred embodiment described above.

I claim:

111. A device for prevention or treatment of thrombosis comprising:
(a) components which cause compression, and
(b) components which create acoustic waves.

112. The device of claim 111 wherein said components which cause compression are comprised of a pneumatic mechanism.

113. The device of claim 112 wherein said pneumatic mechanism is comprised of air bladders.

114. The device of claim 112 wherein said pneumatic mechanism is comprised of pneumatic tubing.

115. The device of claim 112 wherein said pneumatic mechanism is comprised of an air pump.

116. The device of claim 111 wherein said components which cause compression are comprised of an elastic mechanism.

117. The device of claim 111 wherein said components which cause compression operate periodically.

118. The device of claim 111 wherein said components which cause compression operate continuously.

119. The device of claim 111 wherein said acoustic waves are of an ultrasonic frequency.

121. The device of claim 111 wherein said components which create acoustic waves create ultrasonic waves.

122. The device of claim 121 wherein said components which create acoustic waves are ultrasound transducers.

123. The device of claim 123 wherein said ultrasound transducers are comprised of piezoelectric materials.

124. The device of claim 111 wherein said device is used in conjunction with thrombolytic medication or anticoagulant medication.

211. A device comprising:
(a) a mechanism to hold acoustic therapy or thrombosis therapy devices against treatment areas.

212. The device of claim 211 wherein said mechanism is comprised of a textile or other malleable surface.

213. The mechanism of claim 212 wherein the textile or other malleable surface is wrapped around the treatment area.

214. The mechanism of claim 212 wherein the textile or other malleable surface is attached by a mechanism comprising hook-and-loop fasteners.

215. The mechanism of claim 212 wherein the textile or other malleable surface is attached by a mechanism comprising buttons.

216. The device of claim 211 wherein the mechanism is comprised of either a freestanding or supported pole to which the acoustic therapy or thrombosis therapy device is attached.
